(19) United States
(12) Patent Application Publication
(10) Pub. No.: US 2026/0232737 A1
(43) Pub. Date: Aug. 13, 2026

(54) METHODS OF INCREASING THERAPEUTIC PROTEIN LEVELS AND/OR IMPROVING CELLULAR FUNCTION WITH AMNIOTIC FLUID COMPOSITIONS

(71) Applicant: Eliksa Therapeutics, Inc., Doylestown, PA (US)

(72) Inventors: Armen A. Karamanian, Newtown, PA (US); Scott Gross, Pennsauken, NJ (US)

(73) Assignee: Eliksa Therapeutics, Inc., Doylestown, PA (US)

(21) Appl. No.: 19/482,732

(22) PCT Filed: Apr. 11, 2024
(86) PCT No.: PCT/IB2024/053572

§ 371 Date: Nov. 7, 2025

Related U.S. Application Data
(60) Provisional application No. 63/458,617, filed on Apr. 11, 2023.

(51) Int. Cl. A61K 35/50; A61K 9/00; A61K 9/06; A61K 31/7105; A61K 38/17; A61K 38/18; A61K 38/39; A61P 17/02

(52) U.S. Cl. CPC A61K 35/50; A61K 9/0048; A61K 9/06; A61K 31/7105; A61K 38/1748; A61K 38/185; A61K 38/39; A61P 17/02

(57) ABSTRACT

Provided herein are methods of increasing one or more therapeutic protein levels in a subject having epidermolysis bullosa (EB) and methods of increasing cell adhesion and/or attachment in an EB subject by administering to the subject a therapeutically effective amount of a pharmaceutical composition comprising amniotic fluid free or substantially free of cells endogenous to the amniotic fluid. The method can increase production of collagen, type VII collagen, collagen alpha-1 (VII) chain, COL7A1 mRNA, laminin, laminin 332, laminin subunit alpha-3, laminin subunit beta-3, laminin subunit gamma-2, LAMA3 mRNA, LAMB3 mRNA, LAMC2 mRNA, decorin, TUBB3 mRNA, tubulin beta 3 class III, and/or functional fragment of any thereof. A method of increasing corneal nerve regeneration in a subject by administering a therapeutically effective amount of a pharmaceutical composition comprising amniotic fluid free or substantially free of cells endogenous to the amniotic fluid is also provided.

### CROSS-REFERENCE TO RELATED APPLICATIONS

This application claims the benefit of priority to U.S. Provisional Application No. 63/458,617, filed on Apr. 11, 2023. Contents of each of the foregoing applications are incorporated herein by reference in their entirety.

### SEQUENCE LISTING

This application contains a Sequence Listing which is submitted herewith in electronically readable format. The Sequence Listing file was created on Apr. 10, 2024, is named “E114015_1040_SL.xml” and its size is 4.56 kb. The entire contents of the Sequence Listing in the XML file are incorporated by reference herein.

### FIELD OF THE INVENTION

The present disclosure relates to methods of increasing levels of therapeutic proteins such as collagen, laminin, and decorin, and improving cellular function in epidermolysis bullosa (EB).

### BACKGROUND OF THE INVENTION

Epidermolysis Bullosa (EB) is a group of rare genetic conditions that result in complications that can affect various parts of the body, including blistering of the skin and mucous membranes. EB is caused by mutations in genes involved in the attachment between or within the layers of epithelium, mucosa and skin. For example, dystrophic EB (DEB), including recessive dystrophic EB (RDEB) is caused by mutations in the COL7A1 gene, which encodes the type VII collagen protein. Junctional EB (JEB) is caused by mutations in the genes that encode the laminin 332 protein, i.e., the LAMA3, LAMB3 and/or LAMC2 genes. Several other genes are involved in different types of EB. The progressive nature of EB leads to scarring and contractures, causing reduced mobility, fusing of the fingers and toes causing mitten deformities, microstomia, significant disability, and skin cancer.

Currently no treatment exists for EB. Accordingly, there is a need for effective treatment of EB. Most investigational approaches focus on gene therapy to replace the defective mutations and other approaches include protein replacement therapy using recombinant proteins.

### SUMMARY OF THE INVENTION

The present disclosure provides methods of increasing level of one or more therapeutic proteins in a subject having epidermolysis bullosa (EB), comprising administering to the subject a therapeutically effective amount of a pharmaceutical composition comprising amniotic fluid substantially free of endogenous cells.

In some embodiments of the method provided herein, production of one or more therapeutic proteins is increased in the subject. In embodiments, the one or more therapeutic proteins are selected from the group consisting of collagen, laminin, decorin, and tubulin. In some embodiments, the method increases level of type VII collagen or functional fragment thereof and/or laminin 332 or functional fragment thereof and/or tubulin comprises tubulin beta 3 class III or functional fragment thereof. In some embodiments, production of COL7A1 mRNA, collagen alpha-1 (VII) chain protein, LAMA3 mRNA, LAMB3 mRNA, LAMC2 mRNA, laminin subunit alpha-3, laminin subunit beta-3, laminin subunit gamma-2, decorin, TUBB3 mRNA, tubulin beta 3 class III, and/or functional fragment thereof is increased in the subject. In some embodiments, the production of COL7A1 mRNA, collagen alpha-1 (VII) chain, LAMA3 mRNA, LAMB3 mRNA, LAMC2 mRNA, laminin subunit alpha-3, laminin subunit beta-3, laminin subunit gamma-2, decorin, TUBB3 mRNA, tubulin beta 3 class III, and/or functional fragment thereof is increased at a site of a chronic and/or acute wound in the subject.

In another aspect, provided is a method of increasing cell adhesion and/or attachment in a subject having epidermolysis bullosa (EB). The method comprises administering to the subject a therapeutically effective amount of a pharmaceutical composition comprising amniotic fluid substantially free of cells endogenous to the amniotic fluid.

In some embodiments, the therapeutically effective amount of the pharmaceutical composition enhances cell adhesion and attachment without altering cell proliferation.

In some embodiments, the method increases cell adhesion and/or attachment independently from cell proliferation.

In another aspect, provided is a method of increasing corneal nerve regeneration in a subject. The method comprises administering to the subject a therapeutically effective amount of a pharmaceutical composition comprising amniotic fluid substantially free of cells endogenous to the amniotic fluid. In some embodiments, the subject has epidermolysis bullosa (EB).

In some embodiments, the composition is substantially free of lanugo and vernix. In some embodiments, the composition is sterile or has been sterilized. In some embodiments, the composition further comprises pieces of (e.g., micronized, homogenized, morselized, lyophilized) amniotic membrane and/or Wharton's jelly. In some embodiments, the method comprises reconstituting the whole or part of the pharmaceutical composition from a lyophilized composition, e.g., amniotic fluid, amniotic membrane, or Wharton's jelly.

In some embodiments, the composition comprises a therapeutically effective amount of protein. The protein can be endogenous and/or exogenous to the amniotic fluid. In some embodiments, the protein is one or more of type VII collagen, keratin, laminin, and decorin. In some embodiments, the composition comprises a therapeutically effective amount of cell-free mRNA. Cell-free mRNA can be endogenous and/or exogenous to the amniotic fluid. In some embodiments, the cell-free mRNA is a transcript or a fragment thereof of one or more genes selected from the group consisting of COL7A1, COL17A, COL17A1, KRT5, KRT14, KLHL24, PLEC, DST, EXPH5, CD151, LAMA3, LAMB3, LAMC2, ITGA3, ITGA6, ITGB4, FERMT1, and DCN. In some embodiments, the composition comprises a therapeutically effective amount of one or more neurotrophins. In some embodiments, the one or more neurotrophins are selected from the group consisting of nerve growth factor, brain-derived neurotrophic factor, and neurotrophin-3.

In some embodiments, the composition further comprises, or is co-administered with, a penetration enhancer. In some embodiments, the composition is allogeneic relative to the subject. In some embodiments, the subject is human. In some embodiments, the composition is administered to the subject topically, subcutaneously, intradermally, intravenously, intracorneally, or intralocularly. In some embodiments, the subject has a corneal wound, and the composition is administered topically at the site of the corneal wound. In some embodiments, the composition is formulated as eye drops. In some embodiments, the composition is formulated as skin gel.

In some embodiments, the compositions of the present disclosure, including but not limited to decorin, modulate the transforming growth factor (TGF) signaling pathway. In some embodiments, the method provided herein facilitates wound healing. In some embodiments, the method provided herein prevents, alleviates, or treats one or more signs, symptoms, or conditions associated with EB in the subject. In some embodiments, the one or more signs, symptoms, or conditions are selected from the group consisting of pain, pruritus, blisters, keratoderma, granulation, erosion, ulceration, pseudosyndactyly, open wounds, tissue scarring, tissue fibrosis, corneal opacification, corneal scarring, corneal ulcerations, corneal abrasions, blepharitis, ectropion, symblepharon, pterygium, caries, dilated cardiomyopathy, hypoalbuminemia, failure to thrive, muscular dystrophy, osteopenia, osteoporosis, and post-streptococcal glomerulonephritis. In specific embodiments, the composition alleviates or treats corneal opacification in the subject.

In one aspect, provided herein is a method of increasing production of one or more therapeutic proteins (e.g., collagen, laminin, decorin, tubulin) in a subject having epidermolysis bullosa (EB), said method comprising administering to the subject a therapeutically effective amount of a sterile pharmaceutical composition comprising amniotic fluid that contains no cells.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 depicts migration of primary fibroblasts harvested from RDEB patients (“EB-Fibroblasts”) treated with the acellular amniotic fluid composition (“acAF”) and untreated control at 0, 24, 48, and 120 hours after initiation of treatment.

FIG. 2A depicts the gap area distance (fold over time 0) following treatment with the acellular amniotic fluid composition (or no treatment) starting at time 0. FIG. 2B depicts percentage of gap closure following treatment with the acellular amniotic fluid composition (or no treatment) starting at time 0.

FIG. 3 depicts phosphorylated SMAD3 (P-SMAD3) immunoblotting of total lysate of fibroblasts derived from a recessive dystrophic epidermolysis bullosa (“RDEB”) subject (“EB-Fibroblasts”) and normal human breast fibroblasts (“Control Fibroblasts”) treated with or without the acAF composition and/or recombinant decorin (“rDecorin”).

FIG. 4 depicts dose-dependent increase of laminin expression in primary JEB cells (referred to as cell 19 and cell 90) by acAF as assessed by Western blotting.

FIG. 5 depicts ultracentrifugation of acAF with a distinct brown pellet at the tube's bottom.

FIG. 6 depicts detection of exosomes in acAF fractions with CD24 as an exosome marker as assessed by Western blot analysis.

FIG. 7 depicts enhanced collagen 7 expression in primary DEB cells (referred to as cell 45 and cell 57) treated with acAF fractions.

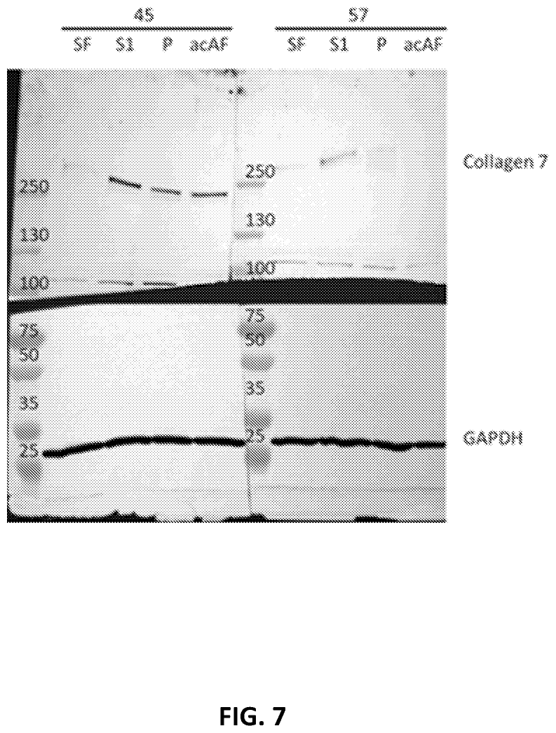

FIG. 8A depicts improved adhesion of DEB cells (indicated by RLU) with acAF treatment. Statistical analysis, revealing a notable difference in adhesion rates among cell types (p=0.0380*), confirms the acAF's impact on cell adhesion. FIG. 8B depicts cell counts (indicated by RLU) across all tested conditions. Significance levels indicated by: *p<0.05, **p<0.01, ***p<0.001, ****p<0.0001.

FIG. 9 depicts a time course of recovery from corneal abrasion in a 9-week old COL7A1 hypomorphic (“C7Hypo”) mouse. Following the injury, the acAF solution (top row) or placebo (bottom row) was administered topically 6 times a day, and slit-lamp photographs were taken daily with fluorescein solution under cobalt blue light to monitor corneal recovery.

FIG. 10 depicts collagen 7 (COL7) mRNA levels (fold change over placebo-treated eyes) in the acAF-treated eyes in the uninjured C7Hypo mice, 9 week old and 13 week old C7Hypo mice with corneal abrasion, and 9 week old wild-type (WT) mice with corneal abrasion.

FIG. 11 depicts tubulin beta 3 class III (TUBB3) mRNA levels (fold change over placebo-treated eyes) in the acAF-treated eyes in the uninjured C7Hypo mice, 9 week old and 13 week old C7Hypo mice with corneal abrasion, and 9 week old wild-type (WT) mice with corneal abrasion.

### DETAILED DESCRIPTION OF THE INVENTION

The present disclosure now will be described more fully hereinafter. The disclosure may be embodied in many different forms and should not be construed as limited to the aspects set forth herein; rather, these aspects are provided so that this disclosure will satisfy applicable legal requirements.

### I. Definitions

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

As used herein, a “subject” is an animal, such as a mammal, including a primate (such as a human, a non-human primate, e.g., a monkey) and a non-primate (such as a cow, a dog, a horse, a sheep, a rabbit, a cat, a rat, or a mouse). In some aspects of the invention, the subject is a human, such as a human having, or at risk of developing, EB. In some aspects, the subject is a pediatric subject, such as a neonate, an infant, or a child. In other aspects, the subject is an adult subject.

As used herein, the term “treating” or “treatment” in the context of treating a disease (e.g., EB) refers to a beneficial or desired result, such as reducing at least one associated sign, symptom, condition, or complication, e.g., pain or pruritus associated with skin conditions, e.g., blisters, in a subject. “Treatment” also refers to a prophylactic treatment, such as prevention of a disease or prevention of at least one sign, symptom, condition, or complication associated with the disease. Accordingly, “treatment” can refer to a reduction in likelihood of developing a disease or associated signs, symptoms, conditions, or complications, or a reduction in severity of a disease or associated signs, symptoms, conditions, or complications relative to a population having the same risk factors and not receiving treatment as described herein. The failure to develop a disease, or a delay in the time to develop associated signs, symptoms, conditions, or complications by days, weeks, months, or years is considered effective treatment. Treatment may require administration of more than one dose of the pharmaceutical compositions comprising acellular AF as described elsewhere herein. “Treatment” can also mean prolonging survival as compared to expected survival in the absence of treatment.

### II. Composition Comprising Acellular Amniotic Fluid

Provided herein are compositions comprising acellular amniotic fluid (“acAF”) for use in treating a subject having EB according to the methods of the present disclosure.

### A. Preparation of Acellular Amniotic Fluid Compositions

Amniotic fluid (“AF”) surrounds a fetus during pregnancy and provides the fetus with a milieu of nutrients and compositions for optimal growth and development. Amniotic membrane, also called amnion, is the inner layer of the placenta and comprises a basement membrane and an avascular stromal matrix. Wharton's jelly is a mucoid connective tissue of the umbilical cord. Human amniotic fluid, amniotic membrane, Wharton's jelly, and associated tissues and compositions can be obtained from tissues and/or body fluids delivered by/from human donors upon informed consent, after delivery of a fetus, placenta, and said tissues and/or body fluids. In specific embodiments, amniotic fluid, amniotic membrane, Wharton's jelly, and/or associated tissues and compositions are donated by healthy human mothers during routine cesarian delivery. Accordingly, obtaining human amniotic fluid, amniotic membrane, Wharton's jelly, and associated tissues and compositions for use in the methods of present disclosure can be carried out without harm (including death) to donors (mother), infants, or newborns, and does not require induced termination of pregnancy. Each donor is tested using FDA approved methods and is found to be non-reactive for Hepatitis B, Hepatitis C, Human Immunodeficiency Virus type 1 & 2, Human T-Lymphotropic Virus type 1 & 2, Syphilis, West Nile Virus and Zika. Non-human amniotic fluid, amniotic membrane, Wharton's jelly, and associated materials can be obtained from non-human animal subjects according to the methods known in the art.

“Acellular amniotic fluid” (acAF) or “cell-free amniotic fluid”, as used herein, refers to amniotic fluid that is substantially free of cells endogenous to amniotic fluid.

“Substantially free of cells” as used herein refers to the status in which the cells are essentially absent, such as containing less than 1-10 cells/ml, or no cells. In specific embodiments, the acAF or acAF composition provided herein comprise no endogenous cells. An “endogenous” cell as used herein in the context of acAF refers to a cell that is contained in crude amniotic fluid obtained from a donor. Without wishing to be bound by theory, crude human amniotic fluid can have about 5×10⁴ cells/ml. The number of endogenous cells in an acAF or an acAF composition provided herein can be less than 0.1% or 0% of that in the crude amniotic fluid obtained from a donor. In specific embodiments, an acAF or an acAF composition contain no cells. “Endogenous cells” as used herein refers to cells that were endogenously present in the crude amniotic fluid as obtained from a donor. Acellular amniotic fluid can be obtained by removing endogenous cells from (crude) amniotic fluid, and can be produced by any means known to those skilled in the art, such as applying centrifugation alone, filtration alone, serial filtration alone, combination of centrifugation and any type of filtration, or combination of centrifugation and serial filtration, to amniotic fluid samples obtained from subjects. In any step of preparation of acellular amniotic fluid, irradiation such as UV light or gamma rays can be included. Acellular amniotic fluid can be sterilized by standard methods, such as filtration, e.g., sterile filtration, irradiation, or combination thereof. In specific embodiments, an acAF is prepared by (i) irradiating with gamma ray crude amniotic fluid obtained from a healthy donor during cesarian delivery upon informed consent; (ii) centrifuging the irradiated amniotic fluid at 1400×g for 15 minutes at 4° C.; (iii) collecting the supernatant and adjusting its pH to 7.4; and (iv) serially filtering the supernatant with a 40 micron and then a 0.2 micron filter. In specific embodiments, an acAF is prepared by (i) centrifuging the crude amniotic fluid amniotic fluid at 1400×g for 15 minutes at 4° C.; (ii) collecting the supernatant and irradiating with UV-C; (iii) adjusting its pH to 7.4; and (iv) serially filtering the supernatant with a 40 micron and then a 0.2 micron filter. acAF from different donors can be combined to created pooled form of acAF. The acAF prepared by this procedure following steps (i)-(iv) above comprise no cells.

In some embodiments, the acellular amniotic fluid is substantially free of lanugo, vernix (also called vernix caseosa), and/or debris, in addition to being substantially cell-free. For example, the acAF provided herein can comprise lanugo, vernix, and/or debris that are less than 2%, less than 1%, less than 0.5%, less than 0.1%, or 0% relative to crude amniotic fluid obtained from a donor. In specific embodiments, the acAF provided herein comprises no lanugo, vernix, and/or debris.

In some embodiments, the pH of the acellular amniotic fluid composition is adjusted to a therapeutically desired level or range of 6.5 to 8.5.

The acellular amniotic fluid or the composition comprising the acellular amniotic fluid can be lyophilized. The lyophilized composition can be reconstituted into a solution by adding a solvent used in the art, e.g., physiological saline. The compositions can be diluted or concentrated. In some embodiments, the composition is sterile or has been sterilized. The composition can be sterilized by subjecting the whole or part of the composition to any sterilization means known in the art, such as filtration, e.g., sterile filtration, irradiation, or combination thereof.

The amniotic fluid compositions provided herein can have therapeutic effects, such as increasing expression of therapeutic proteins (e.g., collagen, COL7, laminin, laminin 332, decorin, tubulin, TUBB3), facilitating cell adhesion and/or attachment, facilitating wound healing, facilitating nerve regeneration, and/or alleviating or preventing one or more symptoms or signs associated with EB. Such therapeutic effects of the amniotic fluid compositions provided herein can be independent of the exosomes or extracellular vesicles contained in the composition, or in crude amniotic fluid.

B. Protein and/or mRNA Compositions

Acellular amniotic fluid compositions of the present disclosure can comprise a therapeutically effective amount of protein and/or mRNA. A “therapeutically effective amount” or “effective amount” as used herein refers to the amount of a composition (e.g., protein, mRNA) effective to produce the intended pharmacological, therapeutic or preventive result, e.g., for treating EB. The therapeutically effective amount of protein and/or mRNA contained in the acAF composition can be endogenous or exogenous. “Endogenous” as used herein refers to being contained in crude amniotic fluid obtained from a donor. “Exogenous” as used herein refers to not being naturally present in crude amniotic fluid, and/or being added to the amniotic fluid or amniotic fluid composition.

In some embodiments, the composition comprises a therapeutically effective amount of protein, which can be exogenous and/or endogenous to the amniotic fluid. The protein in the composition can be one or more of type VII collagen, keratin, and laminin.

In some embodiments, the composition comprises a therapeutically effective amount of mRNA, e.g., cell-free mRNA, which can be exogenous and/or endogenous to the amniotic fluid. “Cell-free mRNA” as used herein refers to extracellular mRNAs existing outside cells. The mRNA can be a transcript or a fragment thereof of one or more genes selected from the group consisting of COL7A1, COL17A, COL17A1, KRT5, KRT14, KLHL24, PLEC, DST, EXPH5, CD151, LAMA3, LAMB3, LAMC2, ITGA3, ITGA6, ITGB4, and FERMT1. In some embodiments, the acellular amniotic fluid compositions comprise one or more of type VII collagen protein, type VII collagen (COL7A1) mRNA, keratin protein, keratin mRNA, laminin protein, and laminin mRNA.

EB can be caused by mutations in one or more genes including but not limited to: COL7A1, COL17A, COL17A1, KRT5, KRT14, KLHL24, PLEC, DST, EXPH5, CD151, LAMA3, LAMB3, LAMC2, ITGA3, ITGA6, ITGB4, and FERMT1. Accordingly, the acellular amniotic fluid compositions of the present disclosure comprising one or more proteins and/or mRNAs discussed above can provide therapeutic compositions that supplement one or more missing, decreased, dysfunctional, or non-functional proteins in EB subjects. Further, the acellular amniotic fluid compositions of the present disclosure can increase (e.g., stimulate) production of one or more mRNAs and/or proteins disclosed herein, e.g., COL7A1, COL17A, COL17A1, KRT5, KRT14, KLHL24, PLEC, DST, EXPH5, CD151, LAMA3, LAMB3, LAMC2, ITGA3, ITGA6, ITGB4, FERMT1, or functional fragment of any thereof, or protein or functional fragment thereof encoded by the gene, and can increase otherwise missing or decreased proteins in EB subjects, e.g., type VII collagen, keratin, or laminin. A “functional fragment” as used herein refers to a fragment of a polynucleotide (e.g., mRNA) or a polypeptide that at least partially retains the function of the full-length polynucleotide or polypeptide. In specific embodiments, administering the acellular amniotic fluid compositions to an EB subject can increase (e.g., stimulate) production of COL7A1 mRNA, collagen alpha-1 (VII) chain protein, and/or functional fragment thereof in the subject, and increase collagen levels (in specific embodiments, levels of type VII collagen or functional fragment thereof) in the subject. For example, administering the acellular amniotic fluid composition can increase levels of collagen, type VII collagen, collagen alpha-1 (VII) chain protein, COL7A1 mRNA, and/or functional fragment of any thereof systemically, or at a site of a chronic and/or acute wound in the subject. In specific embodiments, administering the acellular amniotic fluid compositions to an EB subject can increase (e.g., stimulate) production of LAMA3 mRNA, LAMB3 mRNA, LAMC2 mRNA, laminin subunit alpha-3, laminin subunit beta-3, laminin subunit gamma-2, and/or functional fragment of any thereof in the subject, and increase laminin levels (in specific embodiments, levels of laminin 332 or functional fragment thereof) in the subject. For example, administering the acellular amniotic fluid composition can increase levels of laminin, laminin 332, laminin subunit alpha-3, laminin subunit beta-3, laminin subunit gamma-2, LAMA3 mRNA, LAMB3 mRNA, LAMC2 mRNA, and/or functional fragment of any thereof systemically, or at a site of a chronic and/or acute wound in the subject.

In some embodiments, the acellular amniotic fluid (acAF) compositions comprise a modulator of the TGF signaling pathway, including but not limited to decorin. The compositions can comprise decorin protein and/or a transcript (e.g., mRNA, cell-free mRNA) or a fragment thereof of DCN. Decorin is a protein that belongs to the small leucine-rich proteoglycan family, and can modulate the TGF signaling pathway and/or interact with fibronectin, epidermal growth factor (EGF) receptor, and TGF-β, among other things. “Modulating the TGF signaling pathway”, as used herein, refers to modulating (e.g., increasing or decreasing) the expression or function of molecules that are involved in the TGF signaling, such as TGF-α, TGF-β, EGF receptor, TGF-β receptor, downstream effectors and other TGF-related proteins.

In some embodiments, the acAF composition provided herein, e.g., for use according to the methods provided herein, comprises decorin core protein. “Decorin core protein” as used herein refers to the protein core of a decorin proteoglycan, which comprises a protein core and one or more carbohydrate glycosaminoglycan (GAG) side chains. The decorin core protein can have several distinct structural domains: 1) a short signal sequence of about 16 amino acids; 2) a propeptide of about 14 amino acids; 3) the glycosaminoglycan (GAG) acceptor region with the chondroitin/dermatan sulfate chain substituted at the Ser-4 residue of the mature core protein; 4) a variable cysteine globular domain; 5) a leucine-rich domain with three N-linked oligosaccharide attachment sites; and 6) a carboxyl-terminal globular domain. The mature decorin molecule usually lacks the propeptide domain. The decorin core protein can be about 40 kDa containing about 8-12 leucine-rich repeats (LRR) of about 20-29 residues with leucines.

In some embodiments, the acAF composition comprises decorin in different (e.g., one or more) glycosylation states. Glycosylated decorin can have one or more GAG chains and a size of about 45 to 100 kDa depending on the number of GAG chains and their size. The composition can comprise a plurality decorin subtypes, each having different numbers of GAG chains, different lengths of GAG chains, and/or different sizes of proteoglycan.

The acAF composition can comprise a therapeutically effective amount of one or more neurotrophins. The one or more neurotrophins can be nerve growth factor, brain-derived neurotrophic factor, and/or neurotrophin-3.

### C. Compositions Exogenous to Amniotic Fluid

In some embodiments, the compositions of the present disclosure include exogenous moieties or agents that were not natively present in the amniotic fluid. For example, in some embodiments, the composition further comprises amniotic membrane and/or Wharton's jelly. Amniotic membrane and/or Wharton's jelly can be micronized, homogenized, morselized, or lyophilized prior to being added to the acellular amniotic fluid composition. In some embodiments, the acellular amniotic fluid composition can be lyophilized prior to incorporation of the amniotic membrane and/or Wharton's jelly composition.

Including amniotic membrane or Wharton's jelly to the acellular amniotic fluid compositions of the present disclosure can increase concentrations of therapeutic proteins or mRNA in the composition, e.g., collagen VI, keratin, or laminin.

The compositions exogenous to the amniotic fluid may be a molecule that does not exist in the amniotic fluid. Alternatively, compositions exogenous to the amniotic fluid may be a molecule that can be identified in the amniotic fluid (e.g., type VII collagen protein), but is added to the acellular amniotic fluid composition.

Accordingly, in some embodiments, the composition further comprises a protein composition exogenously added to the acellular amniotic fluid composition. In some embodiments, a recombinant protein of interest or a purified protein of interest can be added to the composition.

In some embodiments, the protein composition comprises one or more of type VII collagen, keratin, laminin, and decorin. In some embodiments, the composition comprises a cell-free mRNA composition exogenously added to the acellular amniotic fluid composition. In some embodiments, the cell-free mRNA composition comprises a transcript or a fragment thereof of one or more genes selected from the group consisting of COL7A1, COL17A, COL17A1, KRT5, KRT14, KLHL24, PLEC, DST, EXPH5, CD151, LAMA3, LAMB3, LAMC2, ITGA3, ITGA6, ITGB4, FERMT1, and DCN.

In some embodiments, the composition comprises a neurotrophin composition exogenously added to the acellular amniotic fluid composition. In some embodiments, the neurotrophin composition comprises protein or mRNA of at least one neurotrophins. In some embodiments, the at least one neurotrophins is selected from the group consisting of: nerve growth factor, brain-derived neurotrophic factor, and neurotrophin-3.

In some embodiments, the concentration of therapeutic molecules, e.g., therapeutic proteins or mRNA, in the composition can be increased or decreased, as needed, relative to the concentration in the amniotic fluid as obtained from donors or subjects. The protein or mRNA concentration can be increased by, for instance, adding the protein or mRNA of interest in the form of amniotic membrane, Wharton's jelly, a recombinant protein, a purified protein, or exogenously prepared nucleic acids. Additionally, or alternatively, the concentration of a protein or mRNA of interest can be decreased relative to the original concentration. The protein or mRNA concentration can be decreased by, for instance, adding an acceptable dilutant, thereby diluting the protein or the mRNA of interest in the composition.

For example, the concentration of type VII collagen can be measured in the acellular amniotic fluid composition, and then an additional amount of amniotic membrane, Wharton's jelly, a recombinant type VII collagen protein, and/or a purified type VII collagen protein can be added to increase the concentration of type VII collagen to a therapeutically desired level. For example, type VII collagen concentration in the acellular amniotic fluid composition can be about 0.3 mg/mL, which can be increased to about 0.6 mg/mL, about 0.9 mg/mL, about 1.2 mg/mL, about 1.5 mg/mL, about 1.8 mg/mL, about 2.1 mg/mL, about 2.4 mg/mL, about 2.7 mg/mL, about 3 mg/mL, or more. Alternatively, type VII collagen concentration can be decreased by adding a vehicle, e.g., physiological saline, to the composition. For example, type VII collagen concentration in the acellular amniotic fluid composition can be about 300 ng/mL, which can be decreased to about 270 ng/ml, about 240 ng/ml, about 210 ng/mL, about 180 ng/mL, about 150 ng/ml, about 120 ng/mL, about 90 ng/ml, about 60 ng/ml, about 30 ng/mL, or less. Similar processes can be applied to increase or decrease the concentration of other therapeutic molecules in the acellular amniotic fluid composition, such as laminin 332 and keratin.

In some embodiments, the pH of the composition comprising acellular amniotic fluid and an exogenous composition can be adjusted to a therapeutically desired level or range, e.g., 6.5 to 8.5. In some embodiments, the composition comprising acellular amniotic fluid and an exogenous composition can be lyophilized. The lyophilized composition can be reconstituted into a solution by adding a solvent used in the art, e.g., physiological saline.

### D. Penetration Enhancers

In some embodiments, the composition further comprises, or is co-administered with, a penetration enhancer. A “penetration enhancer”, as used herein, is a reagent that promotes the penetration of drugs through the epithelial barrier, e.g., a corneal barrier, and change the integrity of the epithelial cell layer. In some embodiments, a penetration enhancer is formulated for ocular delivery. In some embodiments, penetration enhancers that can be included in the composition or co-administered with the composition are include cyclodextrin, dimethylsulphoxide (DMSO), ethylenediaminetetraacetic acid (EDTA), sodium glycocholate and related cholate, Tween 20 (a non-ionic polysorbate surfactant), Brij 35 (polyoxyethylene lauryl ether), saponin, or bile salt. Without wishing to be bound by theory, penetration enhancers such as EDTA and cholates can transiently loosen the tight junctions between adjacent cells of the epithelium, e.g., corneal epithelium. Thus, penetration enhancers, when applied topically, e.g., to the eye, can enhance the delivery of therapeutic molecules, e.g., a protein, a peptide, or mRNA, through the epithelium.

In some embodiments, a penetration enhancer to be included in the composition or to be co-administered with the composition can be a chemical penetration enhancer. A “chemical penetration enhancer”, as used herein, is a reagent that enhances transdermal drug delivery by perturbing the stratum corneum and/or other components of the skin. Chemical penetration enhancers that can be included in the composition or co-administered with the composition via transdermal drug delivery include pyrrolidones, alcohols, esters, water, esters sulfoxides (such as dimethyl sulfoxide) and their derivatives, hydrocarbons, terpenes and derivatives, Azone and its analogs, amides (including urea and its derivatives), fatty acids, surfactants (nonionic, cationic, and anionic), oleodendrimers, ionic liquids, and deep eutectic solvents.

### III. Dosage, Route, and Timing of Administration

A subject (e.g., a human subject having or at risk of developing EB) can be administered a therapeutic amount of pharmaceutical compositions comprising acellular amniotic fluid by any dosage, route or timing that is suitable in the clinical or experimental context. One skilled in the art can determine a dosage, route, or timing for a subject.

For example, the composition can be administered to the subject topically, subcutaneously, intradermally, intravenously, intracorneally, or intraocularly. For example, to treat an EB lesion in the eye, the subject can be administered the acellular amniotic fluid composition intracorneally or intraocularly.

The administration can be one time, or alternatively, repeated, for example, hourly, 4-6 times per day, twice per day, daily, twice per week, weekly, biweekly, or monthly. The administration can be for a prescribed time period, e.g., for one month, two months, three months, four months, five months, six months, one year, longer, or indefinitely. After an initial treatment, the subsequent treatments can be administered less frequently relative to the initial treatment.

The composition can be administered in one dose, or in two or more doses. The number, frequency, or amount of subsequent doses can be dependent on the achievement of a desired therapeutic effect. In some embodiments, the composition is administered to a subject at the frequency and amount required to achieve a therapeutic effect. Further, the subject can be monitored for desired therapeutic effects and unwanted side effects associated with administration of the composition.

The composition can be formulated for delivery to a target organ, e.g., to the eye or to the skin. In some embodiments, the composition is formulated as eye drops for topical administration. In some embodiments, the composition is formulated as skin gel, ointment, or cream. The composition can be administered to the subject in two or more different formulations.

### IV. Increasing Therapeutic Protein Levels in Subjects Having Epidermolysis Bullosa

Provided herein are methods of increasing levels of one or more therapeutic proteins in a subject having EB, or at risk of developing EB. A “therapeutic protein” as used herein refers to a protein that can be beneficial for the cellular, tissue, organ, or body function, including endogenous proteins. Therapeutic proteins include collagen (e.g., collagen VII), laminin (e.g., laminin 332), decorin, and tubulin (e.g., tubulin beta 3 class III). Without wishing to be bound by theory, collagen (e.g., collagen VII), laminin (e.g., laminin 332), decorin, and/or tubulin (e.g., tubulin beta 3 class III) can facilitate wound healing, such as corneal wound healing, or improve corneal opacification in a EB subject. Tubulin (e.g., tubulin beta 3 class III) can facilitate nerve regeneration in a subject. The methods comprise administering to the subject a therapeutically effective amount of a pharmaceutical composition comprising amniotic fluid substantially free of endogenous cells, thereby treating EB. An “effective amount” or “therapeutically effective amount” as used herein refers to the amount of a composition (e.g., acAF composition) effective to increase the amount of the target molecule of interest (e.g., collagen, type VII collagen, laminin, laminin 332, decorin, tubulin, TUBB3) by a certain amount in a subject. One skilled in the art can select the specific percentage, or range of percentages, of increase of a measurable parameter (e.g., the amount of collagen, type VII collagen, COL7A1 protein, collagen alpha-1 (VII) chain protein, laminin, laminin 332, laminin subunit alpha-3, laminin subunit beta-3, laminin subunit gamma-2, LAMA3 mRNA, LAMB3 mRNA, LAMC2 mRNA, decorin, TUBB3 mRNA, tubulin beta 3 class III, functional fragment of any thereof) by which to consider the administration as effective according to the clinical and scientific context.

An “effective amount” or “therapeutically effective amount” can also refer to the amount of a composition (e.g., acAF composition) effective to produce the intended pharmacological, therapeutic or preventive result. For example, in the method of treating a subject having EB provided herein, an effective amount includes an amount effective to reduce one or more signs, symptoms, or conditions associated with EB, e.g., an amount effective to: down-regulate TGF signaling pathway; increase cell proliferation, migration, or adhesion; increase wound healing; reduce or prevent one or more ocular manifestations of EB (e.g., pain, pruritus, corneal opacification, corneal scarring, corneal ulcerations, corneal abrasions, blepharitis, ectropion, symblepharon, pterygium, and loss of vision); reduce or prevent one or more skin manifestations of EB (e.g., pain, pruritus, blisters, keratoderma, granulation, erosion, ulceration, pseudosyndactyly, open wounds, tissue scarring, and tissue fibrosis; reduce or prevent one or more neural manifestations of EB (e.g., neuropathic pain, pruritus, and muscle weakness); and/or reduce or prevent one or more incidents, signs, or symptoms of caries, dilated cardiomyopathy, hypoalbuminemia, failure to thrive, muscular dystrophy, osteopenia, osteoporosis, and post-streptococcal glomerulonephritis. For example, if a given clinical treatment is considered effective when there is at least a certain percentage of reduction in a measurable parameter associated with EB, a therapeutically effective amount of a composition (e.g., acAF composition) for the treatment of EB is the amount necessary to obtain at least the certain percentage of reduction in that parameter. One skilled in the art can select the specific percentage, or range of percentages, of reduction of a measurable parameter by which to consider a treatment as effective according to the clinical and scientific context.

In some embodiments, the method provided herein can increase production of collagen in the subject. “Endogenous” production of collagen as used herein refers to production of collagen by the subject, including cells, tissues or organs of the subject, as opposed to increase in collagen levels by external supply or administration of collagen. For example, the method provided herein can increase production of type VII collagen or fragment thereof in the subject. Without wishing to be bound by theory, type VII collagen is composed of three main domains: a non-collagenous domain (NC-1); a collagenous domain (collagen alpha-1 (VII) chain); and a second non-collagenous domain (NC-2). Collagen alpha-1 (VII) chain is a protein that in humans is encoded by the COL7A1 gene. Collagen alpha-1 (VII) chain is composed of a triple helical, collagenous domain flanked by two non-collagenous domains (NC-1 and NC-2), and functions as an anchoring fibril between the dermal-epidermal junction in the basement membrane. Interactions between the NC-1 domain of collagen VII and several other proteins, including laminin-5 and collagen IV, contribute greatly to the overall stability of the basement membrane. Mutations in COL7A1 cause all types of dystrophic epidermolysis bullosa (DEB), and the exact mutations vary based on the specific type or subtype. In recessively inherited forms of DEB, presence of premature termination codon (PTC)-causing mutations in both alleles results in complete absence of type VII collagen, manifesting with severe mutilating scarring and blistering. Combinations of a PTC-causing mutation with a more subtle missense mutation can result in milder autosomal recessive form of DEB. Most of the dominantly inherited cases of DEB result from glycine substitution mutations in the collagenous domain replacing one of the glycines in the Gly-X-Y repeat triplet sequence. Collectively, the precise degree of severity of DEB reflects the combinations of mutations in COL7A1 and their consequences at the mRNA and protein levels, combined with the effects of modifier genes on the individuals' genetic background and the exposure to environmental trauma. In addition to inherited forms of EB, the acquired form of epidermolysis bullosa (EBA) involves circulating autoantibodies in patients with EBA recognize epitopes in type VII collagen molecule.

In specific embodiments, the method provided herein, to administer the acAF composition, increases production of COL7A1 mRNA, collagen alpha-1 (VII) chain protein, and/or functional fragment thereof in the subject. For example, the method can increase the production of COL7A1 mRNA, collagen alpha-1 (VII) chain, and/or functional fragment thereof at a site of a chronic and/or acute wound in the subject. Expression levels (e.g., mRNA levels) of the COL7A1 gene are measured by any standard methods for measuring mRNA levels of a gene, including quantitative RT-PCR, northern blot, and serial analysis of gene expression (SAGE). Expression levels of collagen alpha-1 (VII) chain are measured by any standard methods for measuring protein levels, including western blot analysis, ELISA, and dot blot analysis.

In some embodiments, the method provided herein can increase production of laminin, decorin, and/or tubulin in the subject. “Endogenous” production of laminin, decorin, and/or tubulin as used herein refers to production of laminin by the subject, including cells, tissues or organs of the subject, as opposed to increase in laminin levels by external supply or administration of laminin. For example, the method provided herein can increase production of laminin 332 or TUBB3 or fragment thereof in the subject. Without wishing to be bound by theory, laminins are a family of large glycoproteins present in various types of basement membranes, which plays important roles in tissue construction and regulation of cellular functions. The laminin molecules consist of three subunits (or chains) of alpha, beta, and gamma, linked by disulfide bonds to form a cross-shape structure. Laminin 332, also referred to as laminin 5, is composed of three subunits: laminin subunit alpha-3, laminin subunit beta-3, and/or laminin subunit gamma-2, each encoded by the LAMA3 gene, the LAMB3 gene, and the LAMC2 gene, respectively. Laminin 332 is an essential component of the dermal-epidermal junction, a specialized basement membrane zone that attaches the epidermis to the dermis and thereby provides skin integrity and resistance to external mechanical forces. Mutations in the laminin subunit encoding genes, e.g., LAMA3, LAMB3, and LAMC2 gene cause junctional epidermolysis bullosa (JEB) characterized by diminished dermal-epidermal adhesion, skin fragility, mechanically induced blistering, and chronic wounds. JEB can include two subcategories: JEB-Herlitz (JEB-H), in which extreme fragility of the skin and mucous membranes usually leads to death within the first years of life; and milder forms collectively called JEB-other or non-Herlitz. JEB-H is caused by loss-of-function mutations in LAMA3, LAMB3 and LAMC2, leading to complete loss of laminin 332. JEB-other is associated with mutations in the above three genes or in COL17A1, the gene coding for collagen XVII, a binding ligand of laminin 332. Rare cases of JEB are associated with integrin α6β4 deficiency and result in JEB with pyloric atresia.

In specific embodiments, the method provided herein, to administer the acAF composition, increases production of LAMA3 mRNA, LAMB3 mRNA, LAMC2 mRNA, laminin subunit alpha-3, laminin subunit beta-3, laminin subunit gamma-2, and/or functional fragment of any thereof in the subject. For example, the method can increase the production of LAMA3 mRNA, LAMB3 mRNA, LAMC2 mRNA, laminin subunit alpha-3, laminin subunit beta-3, laminin subunit gamma-2, and/or functional fragment of any thereof at a site of a chronic and/or acute wound in the subject.

Expression levels (e.g., mRNA levels) of the laminin subunit genes (e.g., LAMA3, LAMB3, LAMC2) are measured by any standard methods for measuring mRNA levels of a gene, including quantitative RT-PCR, northern blot, and serial analysis of gene expression (SAGE). Expression levels of laminin subunits (e.g., laminin subunit alpha-3, laminin subunit beta-3, laminin subunit gamma-2) are measured by any standard methods for measuring protein levels, including western blot analysis, ELISA, and dot blot analysis.

The composition can be allogeneic relative to the subject. The subject can be any animal having EB or suspected to have EB. In specific embodiments, the subject is human. For example, the methods of present disclosure comprise treating a human subject having EB, or at risk of developing EB, comprising administering to the subject a therapeutically effective amount of a pharmaceutical composition comprising human amniotic fluid free or substantially free of endogenous cells.

In some embodiments, the compositions of the present disclosure, in addition to increasing production of COL7A1 mRNA, collagen alpha-1 (VII) chain, type VII collagen, collagen, LAMA3 mRNA, LAMB3 mRNA, LAMC2 mRNA, laminin subunit alpha-3, laminin subunit beta-3, laminin subunit gamma-2, laminin 332, laminin, and/or functional fragment of any thereof, supplement or replace one or more protein defective or absent in cells, tissues, or organs of subjects with EB. The protein or proteins supplemented or replaced can be one or more of type VII collagen, keratin, laminin, decorin, or neurotrophin. Similarly, in some embodiments, the compositions of the present disclosure, in addition to increasing production of COL7A1 mRNA, collagen alpha-1 (VII) chain, type VII collagen, and/or collagen, supplement or replace mRNA defective or absent in cells, tissues, or organs of subjects with EB. mRNA supplemented or replaced can be mRNA of one or more of COL7A1, COL17A, COL17A1, KRT5, KRT14, KLHL24, PLEC, DST, EXPH5, CD151, LAMA3, LAMB3, LAMC2, ITGA3, ITGA6, ITGB4, FERMT1, DCN, or variants thereof. In some embodiments, the compositions comprise therapeutic cell-free mRNA, and it is taken up by cells of subjects with EB for de novo production of proteins defective or missing in EB, i.e., causing cells to produce a replacement protein for the protein that has been defective or missing.

In some embodiments, the acellular amniotic fluid compositions modulate the TGF signaling pathway in a subject, which can result in increase or decrease in levels of TGF-α, TGF-β, and/or other TGF-related proteins in cells, tissues, body fluids, or organs of the subject. The TGF-β pathway is activated (e.g., upregulated) in some EB patients. Without wishing to be bound by theory, activation of the TGF-β pathway is an independent modulator (exacerbator) of the clinical severity of EB, for example independent from the type VII collagen amount in the patient (Chacón-Solano et al. 2022 Matrix Biol. 111:189-206; Nyström et al. 2015 EMBO Mol. Med. 7 (9): 1211-1228; Odorisio et al. 2014 Human Mol. Genet. 23:15; 3907-3922). In some embodiments, administration of the acellular amniotic fluid composition provided herein down-regulates (e.g., attenuates, decreases) the TGF signaling pathway as compared to a control without administration of the acellular amniotic fluid composition. Effects of acellular amniotic fluid on the TGF signaling pathway can be assessed by the standard methods such as those described in Odorisio et al. 2014 Human Mol. Genet. 23:15; 3907-3922. For example, human corneal fibroblasts can be cultured in standard cell culture media supplemented with 10% FBS (fetal bovine serum) until confluency. Then, the media is replaced with test media comprising the acellular amniotic fluid composition at varying dilutions in PBS (incrementally from 1% composition with 99% PBS to 99% composition with 1% PBS), and the cells are incubated at 37° C. for 0, 3, 6, 12, 24 and 48 hours. After the incubation period, TGF-α, TGF-β, ACTA2/α-SMA, SERPINE1/PAI-1, BMP2K, decorin, TGF-β R2, ZEB1, IL7, MMP3, DKK2, tenascin-C, and/or other TGF related proteins in the total cell lysates can be measured or in the supernatant by standard methods, such as ELISA or western blot. The amount of phosphorylated Smad3, phosphorylated Smad2, phosphorylated p38, phosphorylated ERK1/2, phosphorylated AKT, and their ratios over the unphosphorylated counterpart can be measured in total cell lysates by immunoblotting, quantified by densitometry and normalized to GAPDH, as the indicator of the TGF-β pathway activity. Additionally, the expression of TGF-α, TGF-β, ACTA2/α-SMA, SERPINE1/PAI-1, BMP2K, decorin, TGF-β R2, ZEB1, IL7, MMP3, DKK2, tenascin-C, and/or other TGF related mRNA molecules can be quantified by standard methods, such as mRNA isolation, cDNA synthesis, and subsequent real-time PCR quantification. TGF-β receptor I inhibitor SB431542 can be included in the assay as a control.

The compositions of the present disclosure can be used to provide prophylactic, palliative or therapeutic relief to signs or symptoms of EB according to the methods of the present disclosure. In some embodiments, the methods of the present disclosure prevent, alleviate, or treat one or more signs, symptoms, or conditions associated with EB. Accordingly, the methods provided herein can be used to provide prophylactic, palliative, or therapeutic relief to signs or symptoms of EB including but not limited to of pain, pruritus, blisters, keratoderma, granulation, erosion, ulceration, pseudosyndactyly, open wounds, tissue scarring, tissue fibrosis, corneal scarring, blepharitis, ectropion, symblepharon, pterygium, loss of vision, caries, dilated cardiomyopathy, hypoalbuminemia, failure to thrive, muscular dystrophy, osteopenia, osteoporosis, and post-streptococcal glomerulonephritis. In some embodiments, the compositions and methods of the present disclosure prevent, alleviate, or treat one or more ocular manifestations of EB, such as corneal opacification, corneal scarring, corneal ulcerations, corneal abrasions, blepharitis, ectropion, symblepharon, pterygium, and loss of vision. Additionally or alternatively, the compositions and methods of the present disclosure can prevent, alleviate, or treat one or more skin manifestations of EB, such as blisters, keratoderma, granulation, erosion, ulceration, pseudosyndactyly, open wounds, tissue scarring, and tissue fibrosis. Additionally or alternatively, the compositions and methods of the present disclosure can prevent, alleviate, or treat one or more neural manifestations of EB, such as neuropathic pain, pruritus, and muscle weakness.

In specific embodiments, the compositions and methods of the present disclosure facilitates wound healing in subjects (e.g., EB subjects), such as wound healing in the skin, the cornea, or mucosal surface. Wound healing, replacement of damaged or destroyed tissue by newly produced tissue or cells, is a biological process in living organisms, such as humans, and is achieved through the steps of hemostasis, inflammation, proliferation, and remodeling (Sorg et al., 2017 Eur. Surg. Res. 58, 81-94). Chronic wounds, often characterized as wounds that remain open for three months or more, cost an estimated $10-20 billion dollars per year for the US healthcare system alone (Sen et al., 2009 Wound Repair Regen. 17, 763-771). Non-healing wounds cause significant morbidity and mortality, the burden of which has been compared to cancer (Armstrong et al., 2007 Int. Wound J. 4, 286-287). Chronic wounds result from disruption to one of four orchestrated phases that normal, acute wound healing undergoes described above: hemostasis, inflammation, proliferation, and remodeling (Sorg et al., 2017 Eur. Surg. Res. 58, 81-94). Dysregulation of any of these steps leads to non-healing ulcers or excessive scarring. Delayed, dysregulated, or impaired would healing, chronic wounds, and/or non-healing wounds are associated with EB and subjects having EB.

The compositions and methods provided herein can facilitate (e.g., increase) wound healing relative to healing of a control wound to which the acellular amniotic fluid composition of the present disclosure is not administered. Would healing can be measured by any standard methods for assessing wound healing in vitro, ex vivo, or in vivo, including but not limited to a scratch assay, a cell proliferation assay, a cell migration assay, a cell detachment assay, a cell adhesion assay, a collagen lattice contraction assay, or functional evaluation of repaired tissue (e.g., tissue integrity). Exemplary evaluation methods that can be used are provided in the present disclosure.

The compositions and methods of the present disclosure can also facilitate cell adhesion and/or attachment in a subject (e.g., an EB subject). The method can include administering to the subject a therapeutically effective amount of a pharmaceutical composition comprising amniotic fluid substantially free of cells endogenous to the amniotic fluid. The therapeutically effective amount of the pharmaceutical composition can be an amount that enhances cell adhesion and attachment without altering cell proliferation. The method provided herein can increase cell adhesion and/or attachment independently from cell proliferation (e.g., without affecting cell proliferation).

The compositions and methods of the present disclosure can also increase corneal nerve regeneration in a subject (e.g., an EB subject or non-EB subject). Without wishing to be bound by theory, administration of amniotic fluid substantially free of cells endogenous to the amniotic fluid to the subject, e.g., at the site of corneal injury, can increase expression of TUBB3. TUBB3 is a marker of the corneal nerve.

In some embodiments, the composition of the present disclosure, i.e., the acellular amniotic fluid composition, is administered in combination with a second therapy known to be effective in treating EB or preventing, alleviating, or treating one or more signs, symptoms, conditions, or complications associated with EB. The acellular amniotic fluid composition may be administered before, after, or concurrent with the second therapy.

The second therapy may be an additional therapeutic agent. The acellular amniotic fluid composition and the additional therapeutic agent can be administered in combination within the same composition, or co-administered as separate compositions. In some embodiments, the additional therapeutic agent is an anti-inflammatory agent, a pain medication, or an antibiotic. The acellular amniotic fluid composition can be administered in conjunction with a second therapy. Exemplary combination therapies include debridement, skin graft, and gene therapy.

### EXAMPLES

The following examples are offered by way of illustration and not by way of limitation.

### Example 1: Type VII Collagen and Decorin Concentrations in Acellular Amniotic Fluid Samples

Acellular amniotic fluid was obtained from five human donors not having EB. The concentrations of type VII collagen and decorin in the five acellular amniotic fluid samples were measured by commercially available ELISA kits according to the manufacturer's instructions.

As shown in Table 1, the type VII collagen concentrations in acellular amniotic fluid samples ranged from approximately 77 ng/mL to approximately 987 ng/mL, with the average concentration of 335 ng/mL. Dystrophic epidermolysis bullosa (DEB) is caused by mutations in the gene encoding the type VII collagen protein. The data demonstrates that acellular amniotic fluid compositions can be used to supplement therapeutic proteins, including type VII collagen, in DEB patients. The concentrations of other proteins defective or missing in EB patients, e.g., laminin and keratin, in acellular amniotic fluid can also be measured.

Further, as shown in Table 1, the decorin concentrations in acellular amniotic fluid samples ranged from approximately 9 ng/mL to approximately 58 ng/mL, with the average concentration of 27 ng/mL. Decorin is known to modulate the TGF signaling pathway, among other things, and can play a therapeutic role in EB. The data demonstrates that acellular amniotic fluid compositions can be used to provide therapeutic proteins, including decorin, in EB patients.

TABLE 1
type VII collagen and Decorin Concentrations
in Acellular Amniotic Fluid Samples

| | type VII collagen | Decorin |
|---|---|---|
| Acellular Amniotic | Concentration | Concentration |
| Fluid Sample ID | (ng/mL) | (ng/mL) |
| 1 | 190.08 | 18.7 |
| 2 | 76.82 | 8.6 |
| 3 | 986.53 | 58.3 |
| 4 | 233.79 | 28.1 |
| 5 | 190.39 | 21.4 |

### Example 2: mRNA Levels in Acellular Amniotic Fluid Samples

Levels of mutated or defective cell-free mRNA, responsible for pathogenesis of EB, e.g., COL7A1, LAMA3, LAMB3, LAMC2 were analyzed based on data derived from 98 samples of acellular amniotic fluid collected from subjects without EB.

As shown in Table 2, higher levels of COL7A1, LAMA3, LAMB3, and LAMC2 mRNA (detected using gene-specific probes), were present in acellular amniotic fluid samples, relative to control mRNA (detected using all gene-associated probes). Levels of cell-free mRNA of genes defective or missing in other types of EB, e.g., COL17A, COL17A1, KRT5, KRT14, KLHL24, PLEC, DST, EXPH5, CD151, ITGA3, ITGA6, ITGB4, and FERMT1 can also be analyzed.

The Table 2 data demonstrates that cell-free mRNA that are missing or defective in EB subjects are present at high concentrations in acellular amniotic fluid. Cell-free mRNA can be incorporated into cells of EB subjects leading to de novo production of proteins defective or missing in EB.

TABLE 2
mRNA Levels in Acellular Amniotic Fluid Samples

| | | | Median ± S.D. |
|---|---|---|---|
| | | | (min-max) |
| Gene | Annotation | Note | expression |
| All | — | median summarization | 3.61 ± 1.70 |
| genes | | of the expression of all | (1.84-13.86) |
| | | gene-associated probes | |
| | | in the study | |
| COL7A1 | collagen type VII, | COL7A1 | 8.07 ± 0.38 |
| | alpha 1 chain | | (6.99-8.88) |
| LAMA3 | laminin, alpha 3 | part of laminin-332 | 6.64 ± 0.45 |
| | subunit | (α3 chain) | (5.48-7.93) |
| LAMB3 | laminin, beta 3 | part of laminin-332 | 5.46 ± 0.28 |
| | subunit | (β3 chain) | (4.82-6.23) |
| LAMC2 | laminin, gamma 2 | part of laminin-332 | 5.67 ± 0.35 |
| | subunit | (γ2 chain) | (4.80-6.75) |

### Example 3: Acellular Amniotic Fluid Composition Promotes Wound Healing

### Experimental Methods

Amniotic fluid (crude amniotic fluid) was obtained from a healthy donor during cesarian delivery upon informed consent. Crude amniotic fluid was irradiated with gamma ray and then centrifuged at 1400×g for 15 minutes at 4° C. The supernatant was collected, pH was adjusted to 7.4, and then serially filtered with a 40 micron filter and a 0.2 micron filter, resulting in sterile acAF.

To investigate the effect of the acellular amniotic fluid composition (“acAF composition”) on cell migration, an in vitro scratch assay was conducted as previously described by Pitzurra 2020 J. Peridont. Res. 55:287-295). Briefly, fibroblasts derived from recessive dystrophic epidermolysis bullosa (RDEB) patients (“EB-Fibroblasts”) were seeded at an optimal density for confluency into special migration silicon inserts in serum free media with or without the acAF composition (25% acAF in the serum free media) and incubated overnight (each group in duplicate). To segment the area-of-interest, binary masks were created from the raw phase-contrast images with ImageJ software, and the gap/wound closure percentage was quantified as described in Youssefian et al., 2021 J. Invest. Dermatol. 141 (7): 1754-1764. The migration rate and gap closure percentage were measured at 24, 48, and 120 h after treatment.

### Results

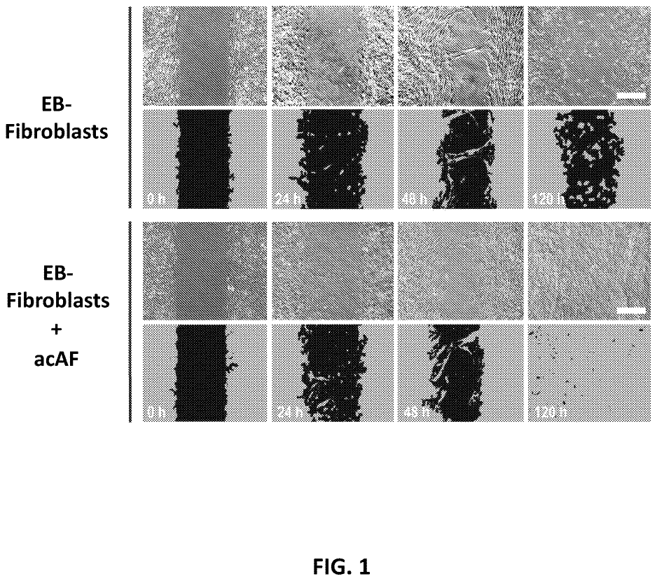

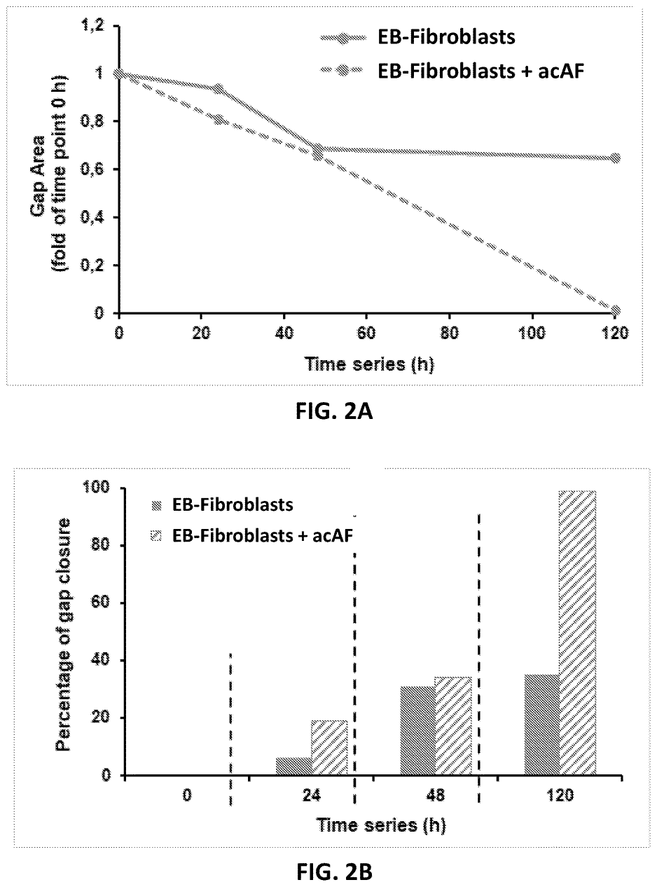

As shown in FIGS. 1, 2A, and 2B, the gap area remained clearly detectable at 120 h in the untreated, control cells. In contrast, closure occurred between 48-120 hours in the acAF composition treated fibroblasts. These data demonstrate the ability of the acAF composition to facilitate fibroblast migration and facilitate wound healing.

### Example 4: Acellular Amniotic Fluid Down-Regulates Transforming Growth Factor (TGF) Signaling Pathway

### Experimental Methods

Sterile acAF was prepared according to the procedure described in Example 3. Effects of an acAF composition on the TGF signaling pathway was assessed by the standard methods such as those described in Odorisio et al. 2014 Human Mol. Genet. 23:15; 3907-3922. Briefly, EB-Fibroblasts derived from RDEB patients described in Example 3, and control fibroblasts derived from healthy subjects (“Control Fibroblasts”) were incubated in serum free media at an optimal density for 24 hours with or without 25% acAF composition or recombinant decorin (positive control). Cells were harvested and lysed at 48 hours. To assess activation of the TGF-β signaling pathway, phosphorylation of Smad3 was quantitated by immunoblotting of total cell lysate. The intensity as well as the ratio of phosphorylated Smad3 and unphosphorylated Smad3 was assessed.

### Results

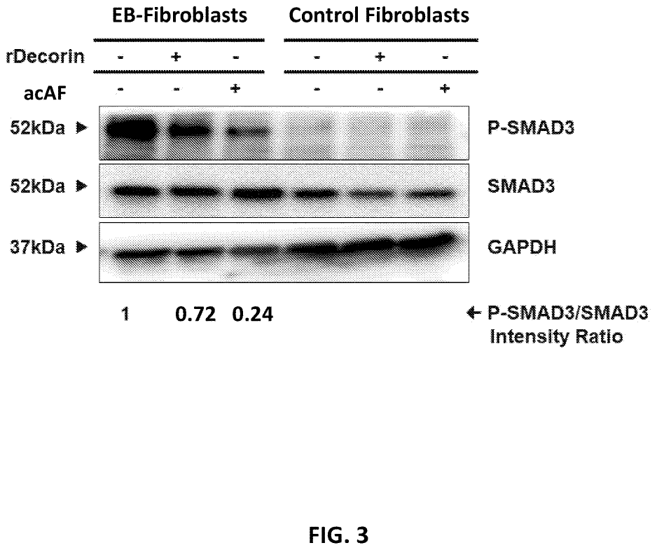

Increased TGF-β signaling, as determined by increased phosphorylation of Smad3, a downstream target of TGF-β, was evident in the primary EB-Fibroblasts RDEB fibroblasts (FIG. 3, lane 1). Addition of recombinant decorin (“rDecorin”), a TGF-β inhibitor, resulted in a mild decrease in smad3 phosphorylation (FIG. 3, lane 2). Importantly, EB-Fibroblasts RDEB primary fibroblasts treatment with a 25% acAF composition for 24 hours showed a significant decrease of Smad3 phosphorylation (FIG. 3, lane 3). Non-detectable phosphorylated Smad3 was observed in Control Fibroblasts, either with or without treatment with the decorin and/or acAF compositions. These data demonstrate that the acAF composition of the present disclosure can down-regulates the TGF signaling pathway. Down-regulation of the TGF pathway can treat EB and ameliorate signs and symptoms associated with EB in a subject independent of the collagen amount in the tissue of the subject.

### Example 5: Functional In Vitro Assays

Proper cell proliferation, migration and attachment are key cellular functions for adequate tissue homeostasis. These cellular functions are typically altered in EB patients resulting in excessive blistering and inadequate wound healing. Cells derived from EB subjects, e.g., fibroblasts, keratinocytes or ocular epithelial cells, exhibit similar altered cellular functions in vitro.

Exemplary in vitro models of EB (e.g., EB cells or EB cell lines) are established as follows. Cells, such as fibroblasts, keratinocytes, and ocular epithelial cells, are harvested from human and non-human subjects having EB, or having a mutation that causes EB, and cultured in vitro. EB cells are also generated by knocking in or knocking down one or more genes or proteins associated with EB, e.g., COL7A1, LAMA3, LAMB3, LAMC2, COL17A, COL17A1, KRT5, KRT14, KLHL24, PLEC, DST, EXPH5, CD151, ITGA3, ITGA6, ITGB4, and FERMT1. From these EB cells, a primary cell culture is established, and optionally passaged. Immortalized EB cell lines are also established. The therapeutic effects of the compositions of the present disclosure are evaluated by cellular assays including the following:

### 1. Cell Proliferation Assay

EB cells and control cells are cultured with the compositions of the present disclosure comprising acellular amniotic fluid having varying concentrations or amounts of a therapeutic molecule, e.g., a protein or mRNA of the gene defective in EB. For example, the cells are cultured in serum free cell culture media comprising acellular amniotic fluid at varying dilutions, e.g., 100%, 75%, 50%, 25%, 10%, 1%, or 0.3% acellular amniotic fluid. Cell proliferation is measured by methods known to those skilled in the art, such as DNA synthesis cell proliferation assays, metabolic cell proliferation assays (e.g., MTT assay), detecting proliferation markers, among others.

### 2. Cell Migration Assay

EB cells are cultured with the compositions of the present disclosure comprising acellular amniotic fluid having varying concentrations or amounts of a therapeutic molecule, e.g., a protein or mRNA of the gene defective in EB. For example, the cells are cultured in serum free cell culture media comprising acellular amniotic fluid at varying dilutions, e.g., 100%, 75%, 50%, 25%, 10%, or 1% acellular amniotic fluid. Cell migration properties are studied by methods known in the art, such as a Boyden chamber, cell culture wounds, scratch assays among others.

### 3. Cell Detachment Assay

EB cells are cultured with the compositions of the present disclosure comprising acellular amniotic fluid having varying concentrations or amounts of a therapeutic molecule, e.g., a protein or mRNA of the gene defective in EB. For example, the cells are cultured in serum free cell culture media comprising acellular amniotic fluid at varying dilutions, e.g., 100%, 75%, 50%, 25%, 10%, or 1% acellular amniotic fluid. The cell detachment properties are measured by standard methods, such as those described in Löffek et al. 2014 PLOS One 9 (2): e87263 and Jacków et al. 2016 J. Invest. Dermatol. 136:1346-1354.

For example, fibroblast or corneal epithelial cells are seeded in cell culture plates, and cultured for 24 hours. Subsequently, cells are washed with phosphate buffer saline (PBS) and treated with trypsin/EDTA (0.05/0.02%) for 10, 6, 4, 2, 1, and 0 minutes followed by another PBS wash. The adherent cells are stained with 0.5% crystal violet in distilled water for 30 minutes, lysed with 1% sodium dodecyl sulfate (SDS), and the percentage of adherent cells can be determined by the measure of the absorbance at 540, 590, or 595 nm using a spectrophotometer. Results are expressed as a percentage relative to 0 minute (trypsin untreated).

Additionally, or alternatively, a centrifugal-force assay is conducted. Briefly, cell culture plates are coated with the compositions of the present disclosure overnight and EB cells are seeded for a period of time ranging from 10 minutes to hours. Subsequently, the cell culture plates are centrifuged at different forces and non-adherent cells are washed with PBS. Adherent cells are fixed, stained with crystal violet, lysed and absorbance of the dye is measured using a spectrophotometer.

### 4. Cell Adhesion Assay

EB cells are cultured with the compositions of the present disclosure comprising acellular amniotic fluid having varying concentrations or amounts of a therapeutic molecule, e.g., a protein or mRNA of the gene defective in EB. For example, the cells are cultured in serum free cell culture media comprising acellular amniotic fluid at varying dilutions, e.g., 100%, 75%, 50%, 25%, 10%, 1%, or 0% acellular amniotic fluid. The cell adhesion properties are measured by standard methods, including those described in Chen et al. 1999 Experim. Cell Res. 249 (2): 231-239. Briefly, cell culture plates are coated with the composition overnight. Fibroblast or corneal epithelial cells are added and allowed to attach for a period of time such as 1.5 hours at 37° C. Subsequently, unattached cells are removed by washing them with PBS. Adherent cells are stained for 15 min with 0.5% crystal violet and washed extensively with distilled water, solubilized in 1% SDS, and quantified by measuring the absorbance.

### 5. Collagen Lattice Contraction Assay

EB cells are cultured with the compositions of the present disclosure comprising acellular amniotic fluid having varying concentrations or amounts of a therapeutic molecule, e.g., a protein or mRNA of the gene defective in EB. For example, the cells are cultured in serum free cell culture media comprising acellular amniotic fluid at varying dilutions, e.g., 100%, 75%, 50%, 25%, 10%, or 1%, or 0% acellular amniotic fluid. The collagen lattice contraction properties are measured by standard methods, including those described in Odorisio et al. 2014 Human Mol. Genet. 23:15; 3907-3922. In brief, collagen solution is produced by mixing acidic-soluble type I collagen (Symatese Biomateriaux, Chaponost, France) 3 mg/ml, a 5-fold concentration of DMEM and a buffer solution (0.05 M NaOH, 2.2% NaHCO₃, 200 mM HEPES) in the ratio 7:2:1. Collagen solution is mixed with cell suspension in serum-free medium, plated in six-well cell culture cluster (Costar; Corning, New York, USA) and gelled at 37° C. for 30 min. The final concentration of collagen can be 2.1 mg/ml. Serum-free DMEM is poured onto the gel to prevent the surface from dehydrating. After 12 h of incubation, the gel is detached from each well and left floating. Surface area of gel samples is measured at detachment (time 0) and after 24 and 48 h. The contraction of the gel can be expressed as percentage of initial lattice area following the formula: A2/A1×100, where A1 is the initial gel area and A2 the area at the observed interval. Three culture plates is used for each experimental group. The assay is conducted on the following experimental groups: (1) gelification performed with 0.25 ng/ml of recombinant human TGF-β1 (R&D Systems) (contraction positive control); (2) gelification performed with 200 nM recombinant human DCN (R&D Systems) and/or in the absence of TGF-1 (0.25 ng/ml) (negative control); and (3) gelification with a 0%, 50%, or 100% acAF composition (experimental group).

### Example 6: Acellular Amniotic Fluid Increases Production of Therapeutic Proteins In Vitro

EB cells are cultured with the compositions of the present disclosure comprising acellular amniotic fluid at varying dilutions. For example, the cells are cultured in serum or serum free cell culture media comprising acellular amniotic fluid at varying dilutions, e.g., 100%, 75%, 50%, 25%, 10%, or 1%, or 0% acellular amniotic fluid for an incubation time (0, 12, 24, 48, 72 or more hours). After the incubation time, cells are washed with serum or serum free media and cultured for a period of time (0, 12, 24, 48 or more hours). At the end of the study, cells and/or media are harvested for qPCR, Western Blot (WB), and immunofluorescence or immunohistochemistry stains to quantitate mRNA and/or protein expression of molecules involved in EB pathology, such as type VII collagen, collagen alpha-1 (VII) chain protein, Col7A1, laminin 332, laminin subunit alpha-3, laminin subunit beta-3, laminin subunit gamma-2, LAMA3, LAMB3, LAMC2, TGF-β, α-SMA, decorin, Ki67, MMP9, tenascin-C, and/or β-III tubulin. Techniques used by those skilled in the art could be used to distinguish the levels between endogenous or exogenous molecules involved in EB pathology. The incubation with acAF composition can increase expression levels of type VII collagen, the collagen alpha-1 (VII) chain protein, Col7A1 gene, laminin 332, laminin subunit alpha-3, laminin subunit beta-3, laminin subunit gamma-2, LAMA3 gene, LAMB3 gene, and/or LAMC2 gene in the EB cells. Production of type VII collagen, laminin 332, and/or functional fragment thereof, e.g., transcription and/or translation of the collagen alpha-1 (VII) chain protein, Col7A1 gene, laminin subunit alpha-3, laminin subunit beta-3, laminin subunit gamma-2, LAMA3 gene, LAMB3 gene, LAMC2 gene, and/or functional fragment thereof can be increased in the EB cells treated with the acAF composition.

### Example 6-1: Acellular Amniotic Fluid Increases Expression of Laminin in EB Fibroblasts

### Experimental Methods

The following primary fibroblasts, obtained from patients with Epidermolysis Bullosa (EB), were purchased from the Coriell Institute Cell Bank:
GM10245, hereafter referred as cell 45, a primary Dystrophic EB (DEB) fibroblast obtained from a 12-year-old Italian female;
GM02857, hereafter referred as cell 57, is a primary DEB fibroblast obtained from a 5-year-old Puerto Rican;
GM10319, hereafter referred as cell 19, is a primary Junction EB (JEB) fibroblast derived from a 5-year-old Caucasian male; and
GM09590, hereafter referred to as cell 90, is a primary JEB fibroblast obtained from a 21-year-old Belizean female.

All cells were received at passage 3 and were cultured in Dulbecco's Modified Eagle Medium (DMEM) supplemented with 10% Fetal Bovine Serum (FBS) and 1% Penicillin/Streptomycin (Pen/Strep), a combination henceforth designated as Full Media. Cell growth and subsequent passages were conducted using methods known to those skilled in the art.

Cells 19 and 90 were seeded in 6-well plates and left to adhere overnight. Subsequently, cells were treated with varying concentrations of acellular amniotic fluid (acAF) for 48 hours. The acAF concentrations were applied in a sequence from left to right: 0%, 5%, 10%, 15%, 20%, 25%, 40%, and 60%.

After incubation, the media was discarded, and the cells were rinsed with PBS before being lysed for protein extraction using RIPA buffer, employing standard techniques familiar to those skilled in the art. Protein concentration was determined through a BCA (Bicinchoninic Acid) protein assay. Subsequently, equal quantities of protein were loaded onto 4-12% Tris-Glycine gradient gels in 1× Tris-Glycine solution, utilizing techniques known to those skilled in the art of Western Blotting.

The gel was immersed in deionized water and gently agitated to shed excess water, followed by transfer onto a nitrocellulose membrane using iBlot2 Transfer Stacks and the iBlot2 apparatus for 10 minutes at 25 V, at room temperature. The membrane was subsequently blocked with 5% Milk (RPI) in either PBS or Tris-Based Saline (TBS) with Tween-20 (PBST/TBST; prepared by combining 100 ml of 10×PBS or TBS, 50 ml of 20× Tween-20, and 850 mL of deionized water) for one hour at room temperature with gentle agitation. It was then washed thrice in PBST/TBST for 10 minutes at room temperature, also with gentle agitation. The membrane was incubated with a 1:1,000 dilution of primary LAMC2 antibody in 5% BSA in PBST/TBST overnight at 4° C. with shaking. After washing thrice for 10 minutes with PBST/TBST at room temperature and gentle shaking, the membrane was incubated with a 1:4,000 dilution of secondary anti-mouse-HRP antibody for one hour at room temperature with gentle agitation. For cell lysates, the membrane was cut at the indicated ladder size; the bottom half was treated with a 1:2,000 dilution of primary GAPDH antibody and a 1:5,000 dilution of secondary anti-rabbit-HRP antibody. Following this, the membranes were washed thrice with PBST/TBST at room temperature with gentle shaking, then developed with a chemiluminescent substrate for approximately 30 seconds. Imaging was conducted using the Chemi Blots setting on the iBright imager for optimal exposure times. Band intensity was analyzed with ImageJ, and LAMC2 band intensities were normalized to the GAPDH loading control to assess relative protein expression across different conditions.

### Results

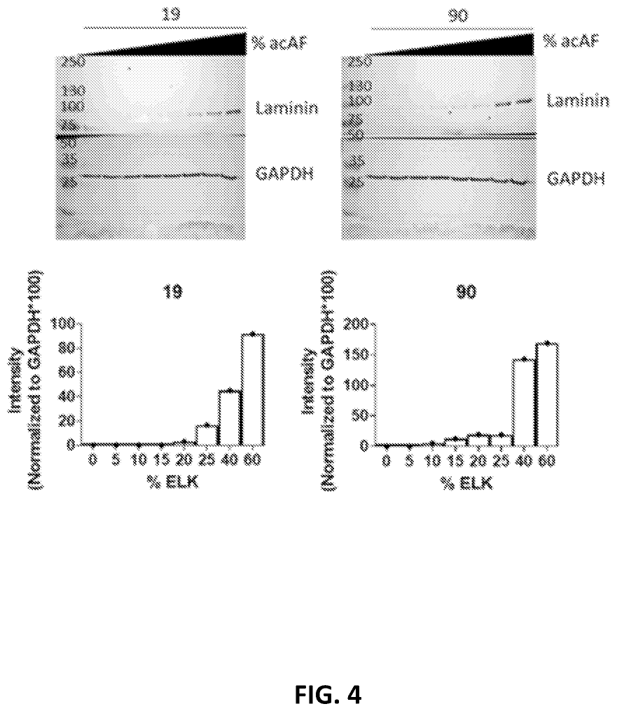

Typically, JEB cells do not express LAMC2. However, as shown in FIG. 4, treatment with acAF resulted in a dose-dependent increase in LAMC2 expression.

### Example 6-2: Analysis of Acellular Amniotic Fluid Fractions

Ultracentrifugation at high Radial Centrifugal Force (RCF) is a commonly utilized method for separating exosomes from solutions. By using the MX120+ ultracentrifuge (ThermoFisher), 7 mL of acAF underwent ultracentrifugation at 53,500 rpm, equivalent to 141,000 RCF (×g), for 24 hours at 4° Celsius. This process yielded distinct exosome-free and exosome-rich fractions.

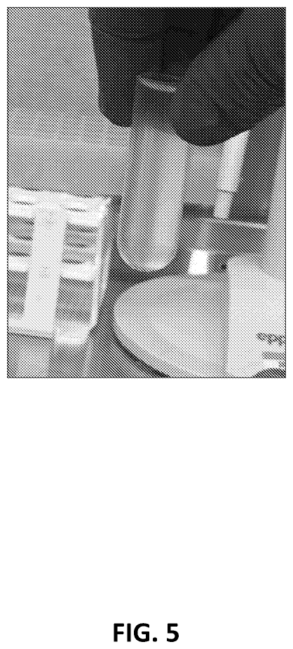

Following ultracentrifugation, the top 1 mL of supernatant was carefully extracted to establish Supernatant Fraction 1 (S1). This procedure was repeated for the subsequent top 1 mL of supernatant, generating sequential fractions S2 through S7. Accordingly, fractions were designated S1 to S7, with S1 denoting the topmost layer of the supernatant and S7 the layer closest to the bottom. The residual pellet, identified as P, was then resuspended in 1-2 mL of DMEM supplemented with 1% Penicillin/Streptomycin, hereinafter referred to as Serum-Free Media. As shown in FIG. 5, after undergoing ultracentrifugation at 53,500 rpm/141,000 RCF (×g) for 24 hours at 4° C., a distinct brown pellet was evident at the bottom.

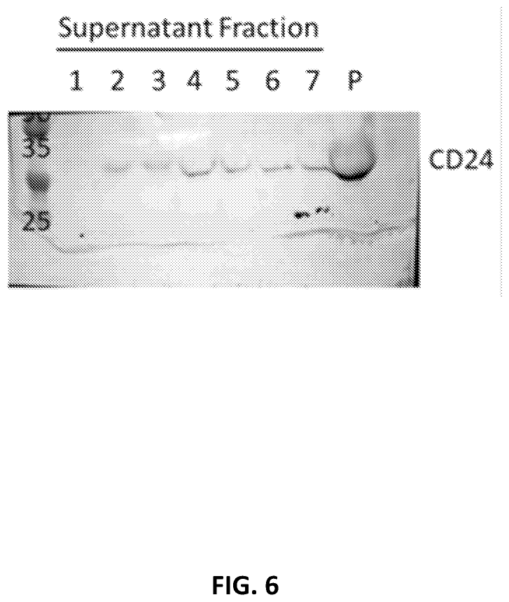

Cluster Differentiation 24 (CD24) is a widely recognized exosome marker in human amniotic fluid. The presence of exosomes in fractions S1 to S7 and the pellet (P) was determined via Western blot analysis targeting CD24. As shown in FIG. 6, fraction S1 showed no exosome presence as indicated by the absence of CD24 in Western blot analysis, whereas exosomes were definitively identified in the pellet fraction (P).

### Example 6-3: S1 and P Fractions of Acellular Amniotic Fluid Increase Expression of Collagen in EB Fibroblasts

### Experimental Methods

Cells 45 and 57 were seeded in 6-well plates and left to adhere overnight. The following day, cells were subjected to a 24-hour treatment with one of the following conditions: serum-free media (SF), 25% S1 fraction (S1) in serum-free media, 25% pellet (P) in serum-free media, or 25% unfractionated acAF in serum-free media. After this treatment period, the media was removed, cells were washed, and cell lysates were prepared as previously described for western blot analysis targeting collagen 7 and GAPDH. Briefly, the membranes were divided into upper and lower segments for incubation: the upper segment was treated with a 1:1,000 dilution of primary anti-collagen 7 antibody, and the lower segment with a 1:2,000 dilution of primary anti-GAPDH antibody, each in a solution of 5% BSA in PBST/TBST. This incubation was performed overnight at 4° C. with gentle agitation. Subsequently, the membranes were washed and treated with the corresponding secondary antibodies. After a series of three washes in PBST/TBST at room temperature with mild agitation, the membranes were developed using a chemiluminescent substrate for around 30 seconds. Imaging was performed using the iBright imager to capture bands at suitable exposure levels.

### Results

The western blot analysis demonstrated that treatment with any fraction of acAF (S1, P, acAF) significantly enhanced the expression of collagen 7 in the EB fibroblasts. Notably, the S1 fraction devoid of exosomes, the pellet fraction abundant in exosomes, and the entire unfractionated acAF sample, all succeeded in elevating collagen 7 expression in EB cells after a 24-hour incubation period. The results indicate that the enhancement of collagen 7 expression by acAF occurs independently of exosomes and is not associated with exosomal activity.

### Example 7: Acellular Amniotic Fluid Increases Adhesion and Attachment of EB Fibroblasts

### Experimental Methods

Cells 45 and 57 were seeded in 96-well plates with full media and left to adhere overnight. The following day, media was removed, cells were washed with pre-warmed sterile PBS, and then cultured in serum-free media containing 0% or 25% acAF for 48 hours. After this period, the media was replaced with pre-warmed sterile PBS. The plate was then securely sealed with parafilm, inverted, and centrifuged at 1,000 g for 10 minutes to apply centripetal force for cell detachment. Subsequently, PBS was exchanged for pre-warmed serum-free media. In parallel, a second plate was prepared but not subjected to centrifugation, serving as a reference to evaluate cell proliferation and establish a baseline for cell quantity prior to centrifugation. Cell viability was assessed using the luminescent cell viability assay, CellTiter-Glo from Promega, following the manufacturer's guidelines. The GloMax plate reader was used to measure Relative Light Units (RLU).

### Results

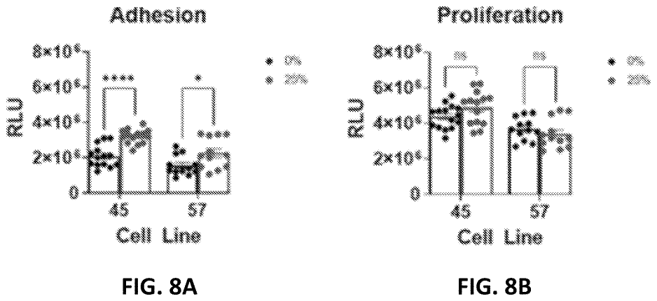

Maintaining proper adherence is a significant challenge for EB cells. As shown in FIG. 8A, acAF significantly improves the ability of EB cells to resist centripetal forces, thereby maintaining their adherence to the culture plate. Comparison of RLU values of the centrifuged plates to those of non-centrifuged control plates show that acAF's effectiveness in boosting EB cell adhesion and maintaining attachment (FIG. 8A) is independent from cell proliferation (FIG. 8B).

### Example 8: Acellular Amniotic Fluid Increases Production of Therapeutic Proteins and Promote Wound Healing in Animal Model of Epidermolysis Bullosa

### Experimental Methods

The compositions of the present disclosure comprising acellular amniotic fluid are administered to animal models of EB, and their therapeutic effects are tested. Several EB animal models are available in various species, including cows, dogs, horses, sheep, cats, rats, and mice. Exemplary EB animal models are described for instance in Bruckner-Tuderman et al. 2010 J. Invest. Dermatol. 130:1485-1488, which is herein incorporated by reference in its entirety. EB animal models include knock-out models, conditional knock-out models, and hypomorphic models.

Amniotic fluid and/or placental materials are donated by healthy mothers during routine cesarian delivery. The collection of the amniotic fluid or placental materials does not entail harm to mothers or newborns, and does not require induced termination of pregnancy. Each donor is tested using FDA approved methods and is found to be non-reactive for Hepatitis B, Hepatitis C, Human Immunodeficiency Virus type 1 & 2, Human T-Lymphotropic Virus type 1 & 2, Syphilis, West Nile Virus and Zika. acAF compositions are prepared as provided in the present disclosure.

The compositions are administered to EB and control subjects as eye drops. Additionally or alternatively, the compositions are delivered to EB and control subjects by topical, subcutaneous, intradermal, intravenous, intracorneal, or intraocular administration. Additionally or alternatively, the compositions are formulated as gels and ointments, and a permeability enhancer is added, and administered to EB and control subjects. In some embodiments, the compositions are combined with a pharmaceutical such as drugs to manage pain or inflammation and co-administered to EB and control subjects.

In one example, a hypomorphic mouse model that produces about 10% of wild-type collagen VII (COL7A1) protein (“C7Hypo”) described in Fritsch et al. 2008 J. Clin. Invest. 8; 118 (5): 1669-1679 was used as an EB animal model. In brief, mice aged 9 and 13 weeks were sedated with inhaled isoflurane, and pre-injury eye conditions were documented using a Topcon slit-lamp camera at 40× magnification. After administering 0.5% proparacaine eye drops for anesthesia, a controlled superficial corneal abrasion was created using a 30G needle, size standardized with 1.8 mm trephine, carefully avoiding the limbal area. The successful removal of the epithelial layer was confirmed using fluorescein eye drops, visualized under cobalt blue light, with the dimensions of the abrasions documented through photographs before initiating treatment. Subsequently, the right eye received 5 μL of acAF, and the left eye was treated with 5 μL Basal Salt Solution (BSS, placebo). Excess fluid was cleaned with single use Weck Cel spears. Treatment was applied four times daily every 2 hours for 10 days or until complete healing occurred (whichever happened first). A drop of 0.3% tobramycin eye drop was applied to each eye as prophylactic antibiotic. Animals were housed in separately over the course of the study. At the end of the study, corneas were harvested and mRNA expression of COL7 and TUBB3 (a corneal nerve marker) was measured. Real-time PCR analysis for COL7 was performed on corneal extracts using methods known to those skilled in the art with the following specific primers: forward TGA TGC TGA CAG ATG AGC TG (SEQ ID NO: 1) and reverse CTC TTA TCA AGT CGC TGT CTC A (SEQ ID NO: 2). Real-time PCR analysis for TUBB3 was performed on corneal extracts using the following specific primers: forward CCT CCG TAT AGT GCC CTT TG (SEQ ID NO: 3) and reverse GTG GAC TTG GAA CCT GGA AC (SEQ ID NO: 4).

qPCR, Western Blot (WB), and immunofluorescence or immunohistochemistry stains to quantitate mRNA and/or protein expression of molecules involved in EB pathology, such as type VII collagen, collagen alpha-1 (VII) chain protein, Col7A1, laminin 332, laminin subunit alpha-3, laminin subunit beta-3, laminin subunit gamma-2, LAMA3, LAMB3, LAMC2, TGF-β, α-SMA, decorin, Ki67, MMP9, tenascin-C, and/or β-III tubulin are further conducted. Techniques used by those skilled in the art could be used to distinguish the levels between endogenous or exogenous molecules involved in EB pathology. Production of type VII collagen, laminin 332, and/or functional fragment thereof, e.g., transcription and/or translation of the collagen alpha-1 (VII) chain protein, Col7A1 gene, laminin subunit alpha-3, laminin subunit beta-3, laminin subunit gamma-2, LAMA3 gene, LAMB3 gene, LAMC2 gene, and/or functional fragment of any thereof is measured.

### Results

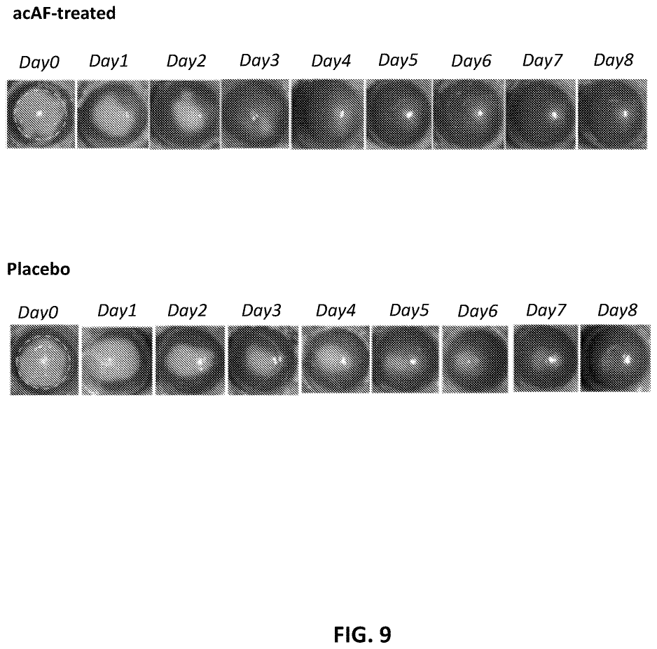

In the cohort of 9 week old mice, acAF was tested in six C7Hypo and in 2 wildtype animals. The corneal abrasion healed quickly in wild-type (WT) mice, reaching the healing endpoint within 24 hours after the injury. The intraocular administration of acAF or placebo did not result in significant difference in healing time in the WT mice. In contrast, acAF significantly reduced healing time in C7Hypo mice compared to the placebo, with corneal abrasions healing in 3 days (versus 8 days for placebo, marking a 2.66 times faster healing rate, p=0.019, FIG. 9). acAF treatment also resulted in significantly lower corneal opacity, measured by the Modified Fantes Score, when compared to placebo treatment.

Similar results were obtained in studies in 13 week old mice. In the cohort of 13 week old mice, acAF was tested in five C7Hypo animals and 2 wildtypes. A similar trend was observed, with acAF accelerating healing in C7Hypo mice (healing in 4 days compared to 7 days for placebo-treated corneas, a 1.75 times faster healing, p=0.002), with no significant impact on wildtype mice treated with acAF or placebo. These findings underscore acAF's potential in enhancing corneal healing specifically in EB-affected corneas, without influencing healing times in normal, wildtype conditions.

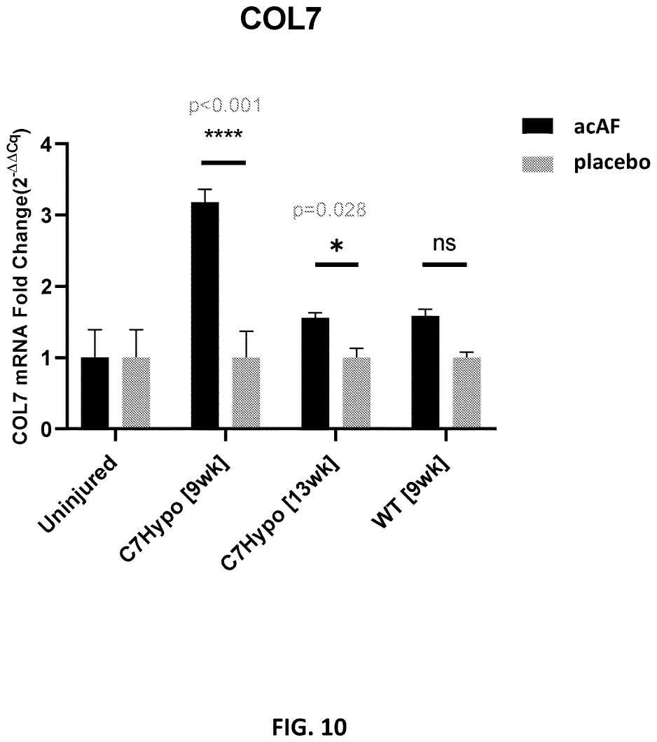

As shown in FIG. 10, real-time PCR analysis for collagen 7 revealed a statistically significant increase in collagen 7 expression in the injured EB animals treated with acAF. In contrast, no significant alterations in collagen 7 levels were detected in the wildtype animals treated with or without acAF, underscoring the specificity of acAF's effect on EB-affected corneas.

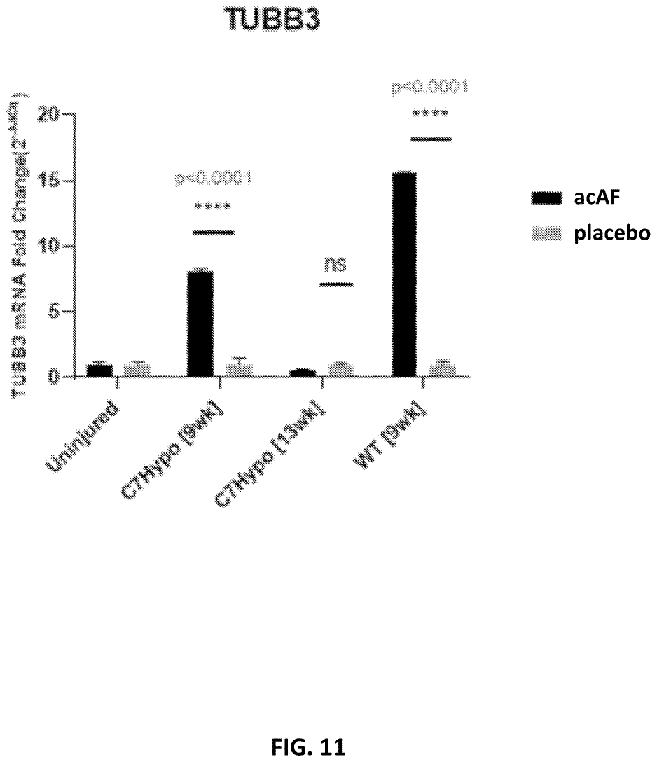

As shown in FIG. 11, real-time PCR analysis for TUBB3, a corneal nerve marker, revealed a statistically significant upregulation of TUBB3 in EB and wildtype animals treated with acAF. This indicates enhanced corneal nerve regeneration following acAF treatment, and highlights the extensive capability of acAF to foster corneal nerve regeneration in both EB-afflicted and non-EB subjects.

Immunofluorescence staining was carried out on corneal sections using standard techniques known to those skilled in the art. Initially, slides were allowed to reach room temperature (~30 minutes) for drying, then fixed in 4% paraformaldehyde (PFA) at room temperature for 15-20 minutes. This was followed by three PBS washes in staining jars, after which a hydrophobic barrier was created around each sample on the slide using an Elite Mini PAP Pen. The slides were blocked with 2% Bovine Serum Albumin (BSA) freshly prepared, for 1 hour at room temperature. Primary antibodies, diluted in 1% BSA, were applied and the slides incubated overnight at 4° C. The antibodies used included collagen 7 (Sigma, catalog #ZRB1507, 1:100 dilution), tubulin 3 (Sigma, catalog #AB15708A4, 1:100 dilution), and mouse decorin (R&D Systems, catalog #AF1060, 1:100 dilution). The next day, after washing the slides three times with PBS, they were incubated with secondary antibodies (diluted 1:2,000) for 2 hours at room temperature, followed by three more PBS washes, and finally mounted with DAPI. Intensity analysis was conducted at 10× magnification, with results normalized against disease-negative controls (DisNeg). acAF treatment was observed to significantly enhance the expression of collagen 7, tubulin 3, and decorin, as shown in Table 3, indicating therapeutic effectiveness of acAF.

TABLE 3
Immunofluorescence analysis of corneas
treated with acAF versus BSS

| | Intensity | | |
|---|---|---|---|
| | Collagen 7 | Tubulin 3 | Decorin |
| acAF | 10.84% | 4.11% | 3.69% |
| BSS (placebo) | 9.61% | 1.85% | 0.62% |

In summary, the results demonstrate the ability of the acAF composition to increase expression of proteins such as COL7, TUBB3, and decorin, and significantly shorten wound healing time and at the wound site in the animal model of EB.

All citations to references, including, for example, citations to patents, published patent applications, and articles, are herein incorporated by reference in their entirety.

The section headings used herein are for organizational purposes only and are not to be construed as limiting the subject matter described in any way.

What is claimed is:

1. A method of increasing production of one or more therapeutic proteins in a subject having epidermolysis bullosa (EB), said method comprising administering to the subject a therapeutically effective amount of a pharmaceutical composition comprising amniotic fluid substantially free of cells endogenous to the amniotic fluid.

2. The method of claim 1, wherein the one or more therapeutic proteins are selected from the group consisting of collagen, laminin, decorin, and tubulin.

3. The method of claim 2, wherein the collagen comprises type VII collagen or fragment thereof, laminin comprises laminin 332 or fragment thereof, and/or tubulin comprises tubulin beta 3 class III.

4. The method of claim 3, wherein production of COL7A1 mRNA, collagen alpha-1 (VII) chain protein, LAMA3 mRNA, LAMB3 mRNA, LAMC2 mRNA, laminin subunit alpha-3, laminin subunit beta-3, laminin subunit gamma-2, decorin, TUBB3 mRNA, tubulin beta 3 class III, and/or functional fragment of any thereof is increased in the subject.

5. The method of claim 3, wherein the production of COL7A1 mRNA, collagen alpha-1 (VII) chain protein, LAMA3 mRNA, LAMB3 mRNA, LAMC2 mRNA, laminin subunit alpha-3, laminin subunit beta-3, laminin subunit gamma-2, decorin, TUBB3 mRNA, tubulin beta 3 class III, and/or functional fragment of any thereof is increased at a site of a chronic and/or acute wound in the subject.

6. A method of increasing cell adhesion and/or attachment in a subject having epidermolysis bullosa (EB), said method comprising administering to the subject a therapeutically effective amount of a pharmaceutical composition comprising amniotic fluid substantially free of cells endogenous to the amniotic fluid.

7. The method of claim 6, wherein the therapeutically effective amount of the pharmaceutical composition enhances cell adhesion and attachment without altering cell proliferation.

8. The method of claim 6 or 7, wherein said method increases cell adhesion and/or attachment independently from cell proliferation.

9. A method of increasing corneal nerve regeneration in a subject, said method comprising administering to the subject a therapeutically effective amount of a pharmaceutical composition comprising amniotic fluid substantially free of cells endogenous to the amniotic fluid.

10. The method of claim 9, wherein the subject has epidermolysis bullosa (EB).

11. The method of any one of claims 1-10, wherein the composition is substantially free of lanugo and vernix.

12. The method of any one of claims 1-11, wherein the composition is sterile or has been sterilized.

13. The method of any one of claims 1-12, wherein the composition further comprises amniotic membrane and/or Wharton's jelly.

14. The method of any one of claims 1-13, wherein the method comprises reconstituting the pharmaceutical composition from lyophilized amniotic fluid, amniotic membrane, and/or Wharton's jelly.

15. The method of any one claims 1-14, wherein the composition comprises a therapeutically effective amount of protein.

16. The method of claim 15, wherein the protein is one or more of type VII collagen, keratin, laminin, and decorin.

17. The method of any one of claims 1-16, wherein the composition comprises a therapeutically effective amount of cell-free mRNA.

18. The method of claim 17, wherein the cell-free mRNA is a transcript or a fragment thereof of one or more genes selected from the group consisting of COL7A1, COL17A, COL17A1, KRT5, KRT14, KLHL24, PLEC, DST, EXPH5, CD151, LAMA3, LAMB3, LAMC2, ITGA3, ITGA6, ITGB4, FERMT1, and DCN.

19. The method of any one of claims 1-18, wherein the composition comprises a therapeutically effective amount of protein or mRNA of one or more neurotrophins.

20. The method of claim 19, wherein the one or more neurotrophins are selected from the group consisting of nerve growth factor, brain-derived neurotrophic factor, and neurotrophin-3.

21. The method of any one of claims 1-20, wherein the composition further comprises, or is co-administered with, a penetration enhancer.

22. The method of any one of claims 1-21, wherein the composition is allogeneic relative to the subject.

23. The method of any one of claims 1-22, wherein the composition is administered to the subject topically, subcutaneously, intradermally, intravenously, intracorneally, or intralocularly.

24. The method of claim 22, wherein the subject has a corneal wound, and the composition is administered topically at the site of the corneal wound.

25. The method of any one of claims 1-24, wherein the composition is formulated as eye drops and/or skin gel.

26. The method of any one of claims 1-25, wherein the composition modulates a transforming growth factor (TGF) signaling pathway.

27. The method of any one of claims 1-26, wherein the composition facilitates wound healing.

28. The method of any one of claims 1-27, wherein the composition alleviates or treats one or more signs, symptoms, or conditions associated with epidermolysis bullosa in the subject.

29. The method of claim 28, wherein the one or more signs, symptoms, or conditions are selected from the group consisting of pain, pruritus, blisters, keratoderma, granulation, erosion, ulceration, pseudosyndactyly, open wounds, tissue scarring, tissue fibrosis, corneal opacification, corneal ulceration, corneal abrasions, corneal scarring, blepharitis, ectropion, symblepharon, pterygium, loss of vision, caries, dilated cardiomyopathy, hypoalbuminemia, failure to thrive, muscular dystrophy, osteopenia, osteoporosis, and post-streptococcal glomerulonephritis.

30. The method of claim 28, wherein the composition alleviates or treats corneal opacification in the subject.

31. A method of increasing production of collagen and/or laminin in a subject having epidermolysis bullosa (EB), said method comprising administering to the subject a therapeutically effective amount of a sterile pharmaceutical composition comprising amniotic fluid that contains no cells.


SEQUENCE LISTING (4 sequences)

SEQ ID NO: 1 - DNA, 20 bp, synthetic construct
  source 1..20: mol_type = other DNA
tgatgctgacagatgagctg

SEQ ID NO: 2 - DNA, 22 bp, synthetic construct
  source 1..22: mol_type = other DNA
ctcttatcaagtcgctgtctca

SEQ ID NO: 3 - DNA, 20 bp, synthetic construct
  source 1..20: mol_type = other DNA
cctccgtatagtgccctttg

SEQ ID NO: 4 - DNA, 20 bp, synthetic construct
  source 1..20: mol_type = other DNA
gtggacttggaacctggaac